(19) United States
(12) Patent Application Publication
(10) Pub. No.: US 2025/0107758 A1
(43) Pub. Date: Apr. 3, 2025

(54) A SYSTEM AND METHOD FOR NON-INTRUSIVE MONITORING AND PREDICTION OF BODY FUNCTIONS

(71) Applicant: Turtle Shell Technologies Private Limited, Indiranagar, Bangalore, Karnataka (IN)

(72) Inventors: Vibhor SARAN, Indiranagar, Bangalore, Karnataka (IN); Vishwa SINGH, Indiranagar, Bangalore, Karnataka (IN); Pavan KAUSHIK, Indiranagar, Bangalore, Karnataka (IN); Gaurav PARCHANI, Indiranagar, Bangalore, Karnataka (IN); Pooja KADAMBI, Bangalore, Karnataka (IN)

(21) Appl. No.: 18/286,198

(22) PCT Filed: Apr. 12, 2023
(86) PCT No.: PCT/IN2023/050358

§ 371 Date: Oct. 9, 2023

(30) Foreign Application Priority Data
Apr. 12, 2022 (IN) 202241021924

(51) Int. Cl. A61B 5/00; A61B 5/11; G16H 50/20

(52) U.S. Cl. CPC A61B 5/7275; A61B 5/1102; G16H 50/20; A61B 5/6892

(57) ABSTRACT

The present invention discloses a system and method for non-intrusive monitoring and prediction of body functions of a user. The system (100) comprises a sensor device (102), a data capturing device (104), a data receiver module (106), an energy spectrum processing platform (108) and user devices (110), which communicate by using a communication network (112). The sensor device (102) comprises a sensor array to capture micro-vibrations of physiological parameters of a user through a surface/mattress under which the sensor device 102 is positioned. Further, the data capturing device (104) is configured to record the communicated micro-voltage data signals in a predefined chronological format for further processing. The energy spectrum processing platform (108) converts the digital time-based BCG data signals into a frequency-based visual output, such as a spectrogram, to accurately view and determine the current body functions, cardiovascular functions, cardiac functions and ejection fraction, such as current ejection fraction, of the user.

### FIELD OF INVENTION

The field of invention generally relates to non-intrusive monitoring and prediction of body functions. More specifically, it relates to a system and method for non-intrusive monitoring and prediction of body functions, cardiovascular functions, cardiac functions and ejection fraction, by converting time-based BCG signals to frequency-based spectrogram, and analyzing the same.

### BACKGROUND

Remote patient monitoring is an emerging area that has seen increased popularity. It employs numerous digital technologies to capture a wide range of biological data from patients by means of multiple sensors including biosensors. The captured biological data can include heart rate, respiratory rate, SPO2, ECG etc. These vitals are typically collected by devices provided to the patients and transmitted to remote patient monitoring platforms by means of a communication network.

Remote patient monitoring systems allow healthcare providers to monitor and assess the health conditions of the patient remotely by continuously monitoring the detected vitals. The ability to predict potential health issues by assessing these collected vitals may help the patients to access better care and treatment in an appropriate time period.

Currently, existing systems do not succeed in predicting potential eventualities for different medical conditions, where the devices are less-or non-intrusive and more comfortable to be used for the patient. Further, current systems also require regular human inputs or intervention to be able to accurately determine changes in body functions such as cardiac output.

Other existing systems have tried to address this problem. However, their scope was limited to predicting the eventualities for particular medical conditions without any visual representation or analysis of the captured sensor data.

Other existing systems have tried to address this problem. However, their scope was limited to using imaging such as Echocardiography or dynamic MRIs can be used as well. However, these systems are not contactless, and cannot function by analyzing a signal alone. Additionally, these system require hygienic facilities which need regular cleaning, and thus are not portable or easy to use in individual settings. Another disadvantage of such systems is that they are expensive and require specialized skilled technicians to operate and interpret any data captured through the same.

Some other systems disclose 3D and 2D display of medical data, however these systems relate to layering of imaging data. Disadvantageously, these systems cannot provide continuous monitoring of a patient or automatic detection of any health issues. Additionally, such systems are also intrusive as they require scanning and imaging systems. Further, these systems often require inputs from a human professional trained and skilled in analyzing such scans and imaging data. Hence, such systems are generally intrusive and non-autonomous, and cannot be used for regular or continuous monitoring of a patient.

Thus, in light of the above discussion, it is implied that there is need for a system and method for continuously monitoring the patient's health status to predict the patient's health status for different medical conditions without human intervention, which is non-intrusive, simple and cost effective, and does not suffer from the problems discussed above.

### OBJECT OF INVENTION

The principal object of this invention is to provide a system and method system for non-intrusive monitoring and prediction of body function.

A further object of the invention is to provide a system and method for converting a user's body vibrations into a visual representation which can be used to determine health and organ status of the user and, in particular, body functions, cardiovascular functions, cardiac functions and ejection fraction of the user.

A further object of the invention is to provide a system and method for non-intrusive monitoring and prediction of cardiovascular functions, cardiac functions and ejection fraction of a user.

Another object of the invention is to provide a system and method for conversion of a mechanical or force-base signal (such as ballistocardiograph (BCG), seismo-cardiographs, and impedance signals) to a visual output such as a spectrogram.

Another object of the invention is to provide a system and method for conversion of a ballistocardiograph (BCG) signal of a user to a visual output such as a spectrogram for determining ejection fraction.

Another object of the invention is to provide a simple, autonomous and cost-effective system and method for non-intrusive monitoring and prediction of body functions, cardiovascular functions, cardiac functions and ejection fraction of a user.

Another object of the invention is to provide a system and method for non-intrusive monitoring and prediction of body functions, cardiovascular functions, cardiac functions and ejection fraction which does not require trained medical practitioners to analyze the BCG signal of the user to determine body functions, cardiovascular functions, cardiac functions and ejection fraction of the user.

Another object of the invention is to provide a system and method for non-intrusive monitoring and prediction of body functions, cardiovascular functions, cardiac functions and ejection fraction which is less or non-intrusive and is very comfortable when implemented in the environment of the user.

Another object of the invention is to provide a system and method for non-intrusive monitoring and prediction of body functions, cardiovascular functions, cardiac functions, and ejection fraction which can also be used in a daily or continuous manner in order to autonomously detect any changes in cardiac output and ejection fraction, without human intervention.

### BRIEF DESCRIPTION OF FIGURES

This invention is illustrated in the accompanying drawings, throughout which, like reference letters indicate corresponding parts in the various figures.

The embodiments herein will be better understood from the following description with reference to the drawings, in which:

FIG. 1A depicts/illustrates a block diagram of a system for non-intrusive monitoring and prediction of body functions, in accordance with an embodiment;

FIG. 1B depicts/illustrates an exemplary placement of a sensor device, in accordance with an embodiment;

FIG. 2 depicts/illustrates a detailed block diagram of components of the system, in accordance with an embodiment;

FIG. 3 depicts/illustrates a detailed block diagram of components of an EF computation unit, in accordance with an embodiment;

FIG. 4A depicts/illustrates an exemplary placement of the sensor device and a data capturing device, in accordance with an embodiment;

FIG. 4B depicts/illustrates an Energy Spectrum for a user with EF>50%, in accordance with an embodiment;

FIG. 4C depicts/illustrates an Energy Spectrum for a user with EF<50%, in accordance with an embodiment;

FIG. 5 depicts/illustrates a method for non-intrusive monitoring and prediction of body functions, in accordance with an embodiment;

FIG. 6 depicts/illustrates a method for non-intrusive prediction of ejection fraction, in accordance with an embodiment; and

FIG. 7 depicts/illustrates a method for non-intrusive monitoring and prediction of body functions of a user, in accordance with an embodiment.

### STATEMENT OF INVENTION

The present invention discloses a system and method for non-intrusive monitoring and prediction of body functions described in the patent application aims to provide a solution for continuous monitoring of users' body functions in a non-intrusive and real-time manner, without requiring human intervention from healthcare professionals. The system comprises a sensor device that can be placed under the user's bed or mattress and a data capturing device that is positioned in the vicinity of the user. The sensor device monitors the user's body functions, including cardiovascular and cardiac functions and ejection fraction, and sends the collected data to the data capturing device for further analysis.

The system also includes an ejection fraction computation unit that determines the ejection fraction of the user's heart based on the collected data. The ejection fraction is a critical parameter that indicates the performance of the heart and helps in diagnosing heart-related diseases. Based on the determined ejection fraction value, the system generates alerts for caretakers, nurses, and healthcare professionals, notifying them about any immediate medical intervention required for the user. This helps in providing timely medical attention to the user and avoiding any adverse health consequences.

The system also includes an energy spectrum computation unit that analyzes the energy spectrum of the collected data to determine the frequency range associated with different body functions. This helps in predicting any abnormalities in the user's body functions, even before the symptoms are visible, and generates alerts for healthcare professionals accordingly.

### DETAILED DESCRIPTION

The embodiments herein and the various features and advantageous details thereof are explained more fully with reference to the non-limiting embodiments that are illustrated in the accompanying drawings and/or detailed in the following description. Descriptions of well-known components and processing techniques are omitted so as to not unnecessarily obscure the embodiments herein. The examples used herein are intended merely to facilitate an understanding of ways in which the embodiments herein may be practised and to further enable those of skill in the art to practice the embodiments herein. Accordingly, the examples should not be construed as limiting the scope of the embodiments herein.

The present invention discloses a system and method for non-intrusive monitoring and prediction of body functions, the system comprises a sensor device that can be placed under the user's mattress or bed to monitor cardiovascular and cardiac functions, ejection fraction, and other body functions in a continuous and real-time manner. The data from the sensor device is captured by a data capturing device, which can be positioned in the vicinity of the user and connected to the sensor device either wirelessly or via a wired connection. The system is capable of providing immediate alerts to healthcare professionals if the user's ejection fraction drops below a certain threshold, indicating that medical intervention is required. The system also includes an EF computation unit that can determine whether the user requires immediate medical intervention based on the Energy Spectrum data collected from the sensor device. Overall, the system is designed to improve the monitoring and care of patients in a hospital or medical care environment, providing timely intervention when necessary and minimizing the need for human intervention from healthcare professionals.

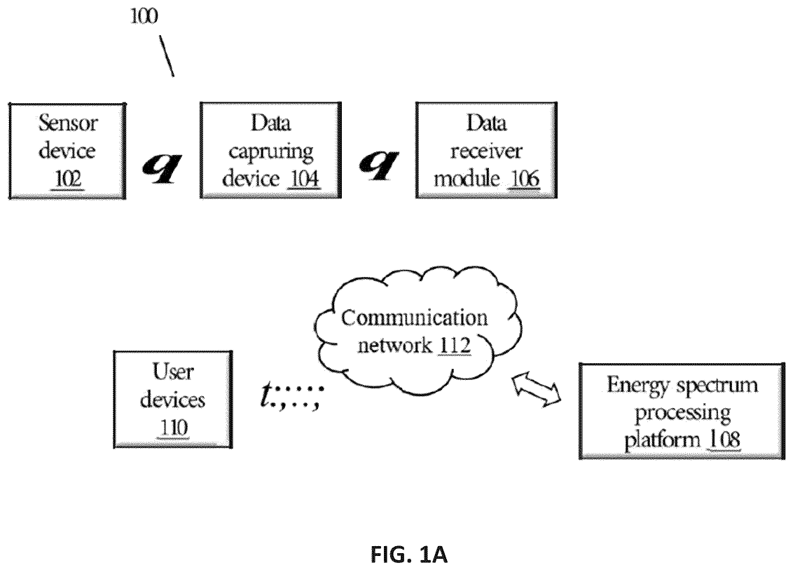

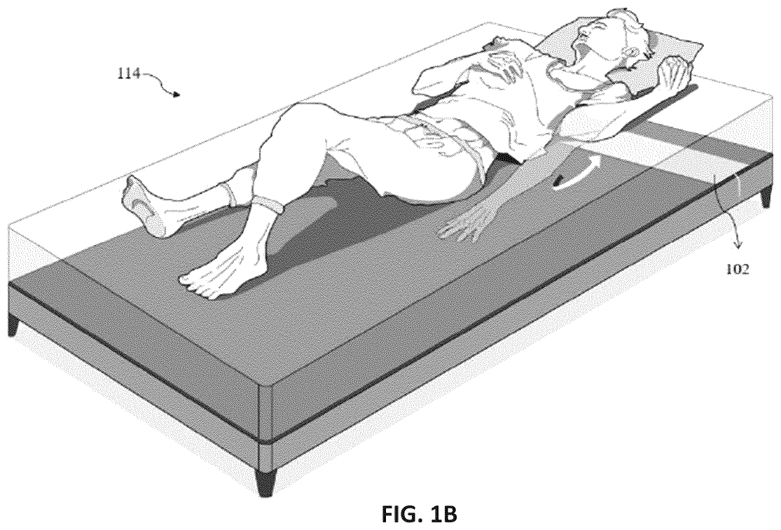

FIG. 1A depicts/illustrates a block diagram of a system 100 for non-intrusive monitoring and prediction of body functions, cardiovascular functions, cardiac functions and ejection fraction in accordance with an embodiment.

In an embodiment, the system 100 comprises at least one sensor device 102, at least one data capturing device 104, at least one data receiver module 106, an energy spectrum processing platform 108 and at least one user device 110, which may communicate by using at least one communication network 112.

In an embodiment, the sensor device 102 is configured to carry out various embodiments of the present invention. The sensor device 102 is placed under a surface or a comfortable medium such as a mattress to ensure maximum comfort of the user, and to ensure that there is no contact between the sensor device 102 and the user being tested/screened/monitored.

In an embodiment, the sensor device 102 comprises at least one sensor array to capture micro-vibrations of one or more physiological parameters of the user's body through the surface under which the sensor device 102 is positioned.

In an embodiment, the micro-vibrations captured by the sensor device 102 comprise one or more mechanical or force-base signals such as ballistocardiograph (BCG), seismo-cardiographs, and impedance signals associated with the physiological parameters of the user's body.

In an embodiment, the BCG signals provide a non-invasive method to measure various physiological motions or process of a user's body, which result from body functions like cardio pulmonary functions like respiratory blockage, effort, fluid buildup in the lungs which include but it is not limited to, cardiovascular functions, cardiac functions, pulmonary function like ejection fraction, cardiac output and so on of the user.

In an embodiment, the term “body functions” as used herein refers to any bodily function that generates vibrations that can be detected by the sensor device 102.

Examples of body functions and corresponding disorders may include, but are not limited to, cardio-pulmonary functions and disorders, cardiovascular disorders, heart rate, blood pressure, ejection fraction, stroke volume, lung capacity, oxygen saturation; pneumonia screening, such as detection of fluid buildup in the lungs, tumor growth in the heart or lungs, valvular disfunction, collapsed lung, arterial blockage etc.

In an embodiment, the BCG signals may be associated with one or more physiological parameters comprising at least one of: cardiac cycles, heartbeat rates, chest movements, heart movements, body movements, stomach functions, lung functions, seizures, tremors, ticks, and respiration, among others.

In an embodiment, the sensor device 102 is configured to convert the captured analog micro-vibrations into digital data signals.

In an embodiment, the analog signals may be converted to the digital signals of any required voltage, as per the requirements of the user or the hardware.

In the depicted embodiment, the digital data signals may comprise micro-voltages in a range of 0.1V to 3.5V.

Further, the sensor device 102 is configured to communicate the digital data signals to the data capturing device 104, which may be positioned in the vicinity of the sensor device 102.

In an embodiment, the data capturing device 104 is configured to record the communicated micro-voltage data signals in a predefined chronological format for further processing. In particular, the data capturing device 104 may calibrate the data signals through amplification, in order to generate an optimized signal with maximum resolution.

Thereafter, the optimized signal is communicated to the data receiver module 106 at regular intervals.

In an embodiment, the data receiver module 106 may comprise a stand-alone module or may be provided within at least one of the energy spectrum processing platform 108 or the user device 110. In an embodiment, the data receiver module 106 is configured to record the communicated micro-voltage data signals in a predefined chronological format for further processing.

In an embodiment, the recorded data signals are processed by various units within the energy spectrum processing platform 108 or by a user application within the user device 110, in order to determine body functions, cardiovascular functions, cardiac functions and cardio-pulmonary of the user.

In an embodiment, as per an advantage of the system, the energy spectrum processing platform 108 may convert the digital BCG data signals (which are time-based signals) into a frequency-based visual output, such as a spectrogram, to accurately view and determine the current body function, such as current ejection fraction, etc, of the user. In an embodiment the visual spectrogram may be representative may represent a stack of frequency over a period of time and the same may be represented in the visual form.

Subsequently, the energy spectrum processing platform 108 may compare the determined current body function, such as ejection fraction with a previously stored data set such as ejection fractions data of the same or set of individuals, in order to predict the future state of the body function of the user or any corresponding disorder-abnormality. Additionally, one or more reports or alerts may be generated, based on the determined current or future state of body function.

In an embodiment, the stored spectrograms may comprise historical data or previously gathered data derived from previous groups of similar users/patients and may represent changes relative to short or long periods of time. Further, the stored spectrograms may comprise one or more tags or descriptions of the health status or issues of the user/patient, related to particular body functions, cardiovascular functions, cardiac functions and Ejection Fraction.

In an embodiment, the functionality of the energy spectrum processing platform 108 may be carried out by similar units in the user device 110.

In an embodiment, the user device 110 may be used by the user to continuously view and monitor their body functions, cardiovascular functions, and cardiac functions such as ejection fraction, and receive one or more reports or alerts related to the same.

In an embodiment, the communication network 112 may comprise wired and wireless communication, including but not limited to, GPS, GSM, WAN, LAN, Wi-fi compatibility, Bluetooth low energy as well as NFC. The wireless communication may further comprise one or more of Bluetooth (registered trademark), ZigBee (registered trademark), a short-range wireless communication such as UWB, a medium-range wireless communication such as WiFi (registered trademark) or a long-range wireless communication such as 3G/4G/5G or WiMAX (registered trademark), according to the usage environment.

FIG. 1B depicts/illustrates an exemplary placement of a sensor device 110, in accordance with an embodiment.

In an embodiment, the sensor device 102 is placed under a surface such as a mattress, cushion, yoga mat, bedding, futon, etc, upon which a user may be seated, laying down, or sleeping. The placement of the sensor device 102 may be aligned towards a sitting, leaning, resting, laying down, or sleeping position of the user.

In an embodiment, the micro-vibrations captured by the sensor device 102 are converted into micro voltages. Thereafter, the micro-voltages are amplified by using a dynamic gain management module. The dynamic gain management module amplifies the micro voltages based on any uncontrollable environmental factors.

In an embodiment, the captured signal may depict variations due to uncontrollable factors such as a user's weight, position and posture, as well as the material, type and thickness of the surface of medium under which the sensor device 102 is positioned.

In an embodiment, such factors may spread the baseline of the signal strength over two or more orders of magnitude. Hence, the dynamic gain management module is used to provide robust data collection with high signal to noise ratio (SNR) with such challenging factors or conditions.

In an embodiment, the gain management module enables dynamic adjustment of the gain of the signal, in order to increase/decrease sensitivity of the signal. Advantageously, the dynamic management of the signal gain enables the capture of data across a wide range of uncontrollable factors.

As an example, dynamic management of the signal gain enables the capture of data from a person of weight 40-120 Kg, through a surface of mattress which is 0-50 cm thick. In an embodiment, the weight of the person and the thickness and/or material of the mattress may vary based on different use-cases.

In an embodiment, the sensor device 102 may be configured to enable appropriate adjustments to the data acquisition rates to capture micro-vibrational signals from one or more other organs such as lungs and stomach; or clinically significant motions such as seizures, tremors, and ticks from the user's body, in case of rapid leg or eye movements.

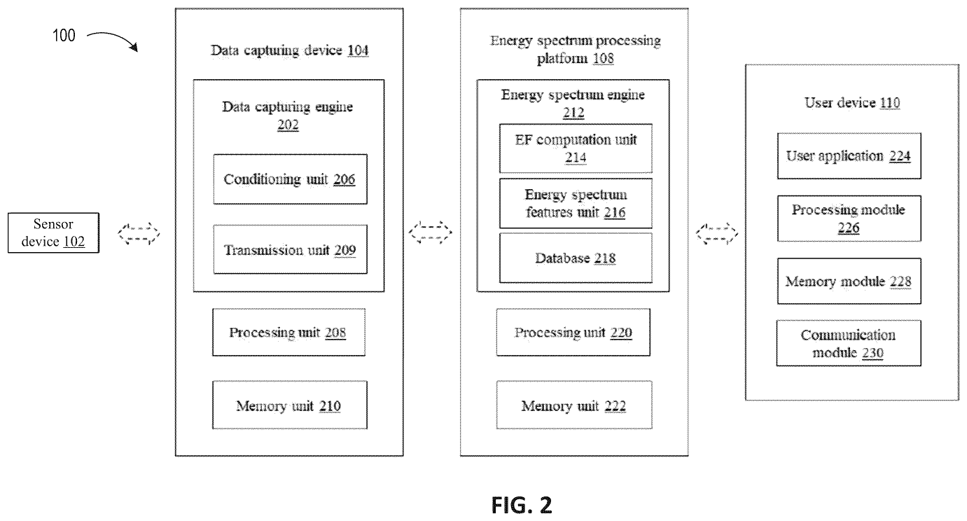

FIG. 2 depicts/illustrates a detailed block diagram of components of the system 100, in accordance with an embodiment.

In an embodiment, the sensor device 102 comprises at least one sensor array comprising one or more of: piezoelectric sensors, and vibroacoustic sensors, among others.

In an embodiment, the sensor array is positioned within a stabilization medium such as a holder, support or a casing. Further, the sensor array is fixed in a pattern and position which is most conducive to obtaining good quality signals from the user.

In an embodiment, the surface or comfortable medium comprises one or more of a mattress, cushion, yoga mat, bedding, futon, etc, upon which a user may be seated, laying down, or sleeping.

In an embodiment, the surface or comfortable medium may comprise one or more material, comprising a spring, foam, air-based mattress/cushion and gel-based mattress/cushion.

In another embodiment, the sensor device 102 may be directly positioned under the user.

In an embodiment, the sensor device 102 may be 2-5 mm thick, and may comprise a strengthened outer cover for protecting and covering inner components of the sensor device 102. The outer cover may comprise a durable material, comprising at least one of plastic, cloth, canvas, mesh, and latex, among others.

In an embodiment, the sensor device 102 comprises an electromagnetic shielding fabric that encases the sensor array. This electromagnetic shielding fabric increases the signal to noise ratio of the detected signal. Advantageously, the electromagnetic shielding fabric prevents corruption of the signal from any erroneous noise from the environment of the sensor device 102.

In an embodiment, the sensor device 102 may be positioned across the body in various configurations of size and spacing.

In an embodiment, the sensor device 102 may be manufactured based on various shapes and sizes, as per different user requirements. Further, different use-cases of the sensor device 102 may comprise different thicknesses and shapes. In an embodiment, the shape of the sensor device 102 may be polygonal, circular, triangular, square, rectangular etc.

As an example, a sensor device 102 placed behind a chair cushion may vary in shape and size from a sensor device 102 used underneath a mattress or a yoga mat, among others.

In an embodiment, the data capturing device 104 comprises a data capturing engine 202, a processing unit 208, and a memory unit 210.

In an embodiment, the processing unit 208 of the data capturing device 104 may comprise one or more of microprocessors, circuits, and other hardware configured for processing. The processing unit 208 is configured to execute instructions stored in the memory unit 210 as well as communicate with at least one of: sensor devices 102, data capturing devices 104, data receiver modules 106, and user devices 110 through an input/output module via a communication module.

In an embodiment, the memory unit 210 of the data capturing device 104 comprises one or more volatile and non-volatile memory components which are capable of storing data and instructions to be executed.

In an embodiment, the data capturing engine 202 is configured to receive the data signals from the sensor device 102, and process the data signals in order to derive optimized data signals.

In an embodiment, the data capturing engine 202 comprises a data acquisition unit 204, a conditioning unit 206, and a transmission unit 209.

In an embodiment, the data acquisition unit 204 is configured to receive one or more micro-voltage digital data signals captured by the sensor device 102.

Further, the data acquisition unit 204 is configured to record or store the received micro-voltage digital signal in a predetermined data format. The pre-defined data recording format may be based on a chronological order format. Further, the data acquisition unit 204 may store the received data signals based on one or more of: user accounts, database segments for different users, different geographical applications, and different applications of the sensor device 102.

In an embodiment, the data acquisition unit 204 is configured to communicate the stored micro-voltage digital data signals to the conditioning unit 206.

In an embodiment, the data acquisition unit 204 is configured to utilize one or more wireless encryption to ensure the communication of secure, encrypted, ordered data is sent to the cloud.

In an embodiment, the conditioning unit 206 is configured to amplify the received micro-voltage digital signals in order to maximize resolution of the digital signals to obtain a desired optimized signal. The conditioning unit 206 is configured to determine the optimized signal for efficient detection of the user's physiological parameters, comprising body functions, cardiovascular functions, cardiac functions and ejection fraction.

In an embodiment, the conditioning unit 206 maximizes the resolution of the digital signals by ensuring prevention of any data loss which may occur due to clipping.

Advantageously, the amplification and maximization of the resolution of the digital signal enables the sensor device 102 to be positioned under a surface or mattress of any thickness, construction or material. Hence, the sensor device 102 may be used in multiple user set-ups and environments.

In an embodiment, the conditioning unit 110 is configured to amplify the micro-voltage digital signal based on strength of the received micro-voltage digital signals.

In an embodiment, the conditioning unit 110 is configured with multiple amplification categories. In an embodiment, the conditioning unit 110 may comprise eight different amplification categories, in order to amplify the micro-voltages between a range of 15x to 2500x, which can cover various body functions, cardiovascular functions, cardiac functions and ejection fraction.

In an embodiment, the conditioning unit 110 is configured to automatically calibrate and select the amplification option based on a sensed proximity of the sensor device 102 to the user, as different applications of the sensor device 102 may position the sensor device 102 at different distances from the body of the user.

Further, the conditioning unit 110 is configured to communicate the optimized amplified signal to the transmission unit 209.

In an embodiment, the transmission unit 209 is configured to transmit the amplified micro-voltage digital signal to one or more of the energy spectrum processing platform 108, a database 118, and the data receiver module 106.

The amplified micro-voltage digital signal may be transmitted to the through the communication network 112.

In an embodiment, the energy spectrum processing platform 108 is configured to convert the digital time-based signals into frequency-based visual outputs such as a spectrogram. Further, the energy spectrum processing platform 108 is configured to determine the current and future body functions, cardiovascular functions, cardiac functions and ejection fraction based on the spectrogram.

Subsequently, the energy spectrum processing platform 108 is configured to generate one or more reports and alerts based on the determined current and future body functions, cardiovascular functions, cardiac functions and ejection fraction.

In an embodiment, the energy spectrum processing platform 108 comprises an energy spectrum engine 212, a processing unit 220, and a memory unit 222.

In an embodiment, the processing unit 220 of the energy spectrum processing platform 108 may comprise one or more of microprocessors, circuits, and other hardware configured for processing. The processing unit 220 is configured to execute instructions stored in the memory unit 222 as well as communicate with at least one of: sensor devices 102, data capturing devices 104, data receiver modules 106, and user devices 110 through an input/output module via a communication module.

In an embodiment, the memory unit 222 of the energy spectrum processing platform 108 comprises one or more volatile and non-volatile memory components which are capable of storing data and instructions to be executed.

In an embodiment, the energy spectrum engine 212 comprises an EF computation unit 214, an energy spectrum features unit 216, and a database 218.

In an embodiment, the EF computation unit 214 is configured to compute the Ejection fraction (EF) of the user. The EF value measures the amount of blood pumped out of the user's heart's lower chambers, called ventricles. The EF provides an insight into the percentage of blood that leaves the left ventricle when a user's heart contracts.

Generally, a normal EF is considered to have a value of 50% or higher. Hence, in case a user is detected to have an EF value which is 50% or lower, it is an indication that the user's heart or their left ventricle may not be pumping out enough blood efficiently. Thus, the EF can be used to determine the cardiac heath of the user.

In an embodiment, the energy spectrum features unit 216 is configured to process the spectrogram by adding one or more layers of filtering and analysis. Thereafter, the energy spectrum features unit 216 may achieve a quantifiable measurement of left ventricular ejection fraction.

In an embodiment, the energy spectrum features unit 216 processes the spectrograms/spectral maps by modifying one or more features comprising color contrast, brightness, relative coloration, and measurable segments in the spectrogram image.

In an embodiment, the energy spectrum features unit 216 processes features comprising at least one of lines, circles, colored pixels, and colored segments, in order to visually assess and interpret the user's vibration data, to represent the physical reality of the user's body's physiological parameters such as body functions, cardiovascular functions, cardiac functions and ejection fraction.

In an embodiment, the database 218 is configured to store one or more micro-voltage digital signals in a predetermined data storage format. The predetermined data storage format may comprise storing one or more datasets in a chronological order format, in the form of micro-voltage digital signal datasets.

In an embodiment of the present invention, the database 118 is configured to transmit the stored micro-voltage digital datasets corresponding to physiological parameters of the subject, to one or more other components of the system 100.

In an embodiment, the database 218 is also configured to save one or more stored spectrograms of body functions, cardiovascular functions, cardiac functions and ejection fractions for various cardiac events and health statuses. These stored spectrograms may be further used for predicting future body functions, cardiovascular functions, cardiac functions and ejection fractions of the user.

In an embodiment, the user device 110 is configured to enable the user to view their body functions, cardiovascular functions, cardiac functions and ejection fraction. In an embodiment, the user device 110 is configured to enable the user to view past, as well as current and predicted body functions, cardiovascular functions, cardiac functions and ejection fraction. The viewable body functions may comprise the ejection fraction of the user.

In an embodiment, the system 100 may comprise as many user devices 110 as required by the users. The user devices 110 may comprise one or more wearable devices, mobile phones, tablets, PDA, smartphones, smart band, smart watch, laptop, computer, etc.

Further, the user device 110 comprises a user application 224, a processing module 226, a memory module 228 and a communication module 230.

In an embodiment, the processing module 226 of the user device 110 may comprise one or more of microprocessors, circuits, and other hardware configured for processing. The processing module 226 is configured to execute instructions stored in the memory module 228 as well as communicate with at least one of: sensor devices 102, data capturing devices 104, and data receiver modules 106, through an input/output module via the communication module 230.

In an embodiment, the memory module 228 of the user device 110 comprises one or more volatile and non-volatile memory components which are capable of storing data and instructions to be executed.

In an embodiment, the user application 224 is configured to provide a user interface and dashboard for the user to view their body functions, cardiovascular functions, cardiac functions and ejection fraction. Further, in case of any determined health issues related to the body functions, cardiovascular functions, cardiac functions or ejection fraction of the user, the user application 224 is configured to provide one or more alerts in the form of vibratory, audio, and visual alerts. Additionally, the user application 224 may be configured to alert one or more healthcare providers and designated contacts of the user.

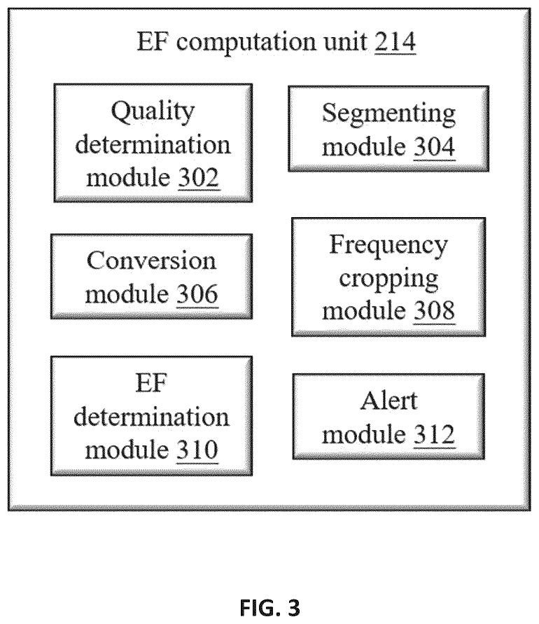

FIG. 3 depicts/illustrates a detailed block diagram of components of the EF computation unit 214, in accordance with an embodiment.

In an embodiment, the EF computation unit 214 comprises a quality determination module 302, a segmenting module 304, a conversion module 306, a frequency cropping module 308, an EF determination module 310 and an alert module 312.

In an embodiment, the quality determination module 302 is configured to verify the quality of the BCG signal based on several quality parameters, and determine one or more portions which pass the quality parameters.

In an embodiment, the quality parameters comprise one or more of good standard deviation, no excessive movement during data capturing, indication of any body event, cardiovascular event, cardiac event, extraction of motion, noise pattern scanning, double counting and sudden jumps above 2 or more standard deviations, among others.

In an embodiment, the segmenting module 304 is configured to create at least one segment from the verified portions of the BCG signal, for further processing.

In an embodiment, the conversion module 306 is configured to convert at least one segment into a Spectrogram or a format that represents the energy distribution of the BCG signal.

In an embodiment, the formats comprise at least one of: root mean square, discrete or continuous wavelet transforms, mel frequency cepstrum, spectral contrast, and Short Term Fourier Transform (STFT), among others.

In an embodiment, the Short Term Fourier Transform (STFT) Spectrogram may be best performing method. Further, the STFT spectrogram is used to visualize sinusoidal frequency and phase content of sections of the BCG signal of the user

In an embodiment, the STFT spectrogram is used to visualize sinusoidal frequency and phase content of sections of the BCG signal of the user.

In an embodiment, the frequency cropping module 308 is configured to determine and clip/crop the STFT spectrogram to retain frequencies comprising valuable information related to the ejection fraction of the user.

In an embodiment, the frequency cropping module 308 may comprise a 0-40 Hz filter as a preprocessing step. In other embodiments, other filter may be used as per user requirements.

In an embodiment, the EF determination module 310 is configured to process the cropped spectrogram, to determine the current ejection fraction, which is helpful in determining the user's health.

In an embodiment, the EF determination module 310 uses at least one algorithm to determine the ejection fraction, comprising at least one statistical or Machine Learning algorithm. The statistical algorithm may comprise at least one of K-means, and Hidden Markov Models.

In an embodiment, the Machine Learning algorithm may comprise at least one of Support Vector Machines, Decision Trees, Random Forest, Artificial Neural Networks, and Convolutional Neural Networks.

In an embodiment, such spectrograms may be compared from multiple data captured from different regions of the user's body. Subsequently, by calculating the decay constant, the overall body functions, cardiovascular functions, cardiac functions or ejection fraction of the user may be determined.

In an embodiment, the calculated decay constant may be invariant across thicknesses and types of multiple mattresses or surfaces under which the sensor device 102 may be placed. Hence, advantageously, the disclosed system provides a reliable way to measure and visualize the change of body functions, cardiovascular functions, cardiac functions and ejection fraction of a single user over a long duration, and hence provides immense benefits for personalized non-intrusive healthcare, which does not require any human intervention.

In an embodiment, the EF determination module 310 is configured to compare the determined body functions, cardiovascular functions, cardiac functions or ejection fraction spectrograms with stored spectrograms in the database 218. The stored spectrograms may comprise historical data derived from previous users/patients. Further, the stored spectrograms may comprise one or more tags or descriptions of the health status or issues of the user/patient, related to particular body functions, cardiovascular functions, cardiac functions and ejection fraction.

Subsequently, based on the comparison, the EF determination module 310 is configured to predict one or more future body functions, cardiovascular functions, cardiac functions or ejection fraction or values for the user.

In an embodiment, the EF determination module 310 is configured to achieve Ejection Fraction Estimating and Bucketing.

In an embodiment, the EF determination module 310 is configured to receive one or more inputs from an admin or a healthcare professional, related to ranges or buckets of EF for the user.

In an embodiment, the one or more ranges or bucket values for bucketing may be used to determine one or more corresponding health statuses of the user.

In an embodiment, the one or more ranges or bucket values may correspond to actual Ejection Fraction values of 0-100%. Further, there may be at least 2 or more buckets of severity, and, in an embodiment, the EF determination module 310 is configured to detect three clinical buckets of severity.

In an embodiment, the EF determination module 310 is configured to determine or detect an Ejection Fraction of a user which is less than 30%. In this case, the Ejection Fraction may indicate that the user has a severe health issue such as a serious dysfunction or heart failure. Additionally, the EF determination module 310 is configured determine normal or abnormal Heart Failure, based on the detected Ejection Fraction.

In an embodiment, the alert module 312 is configured to generate one or more alerts, based on the current and future ejection fractions and decay constant calculated from the user's spectrogram. In an embodiment, the alert may comprise at least one of, a text message alert, mail alert, audio alert, and visual alert, among others.

In an embodiment, the alert may be communicated to one or more users, user devices 110, designated contacts of a user, healthcare professionals, doctors, nurses, and clinicians, among others.

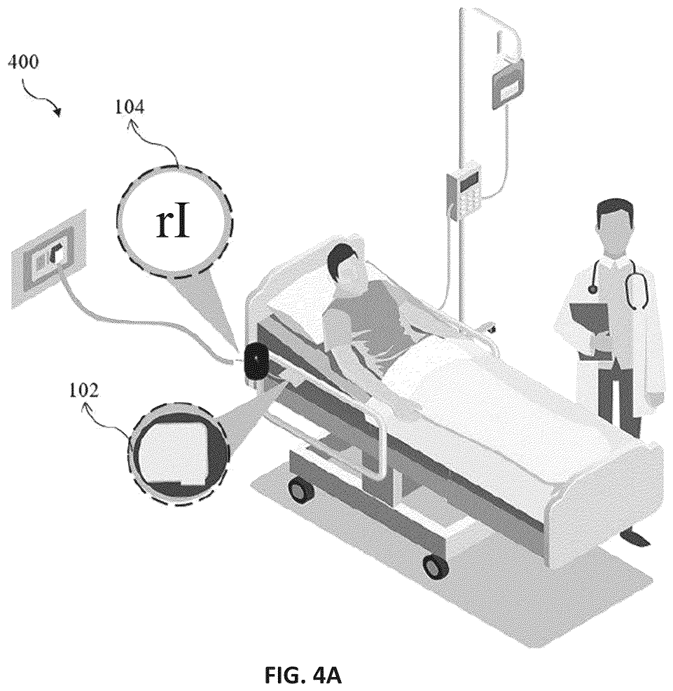

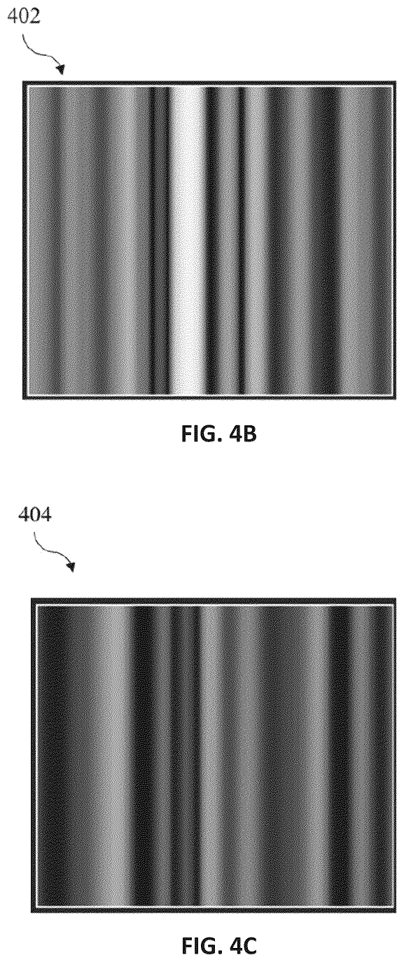

FIG. 4A depicts/illustrates an exemplary placement of the sensor device 102 and the data capturing device 104, in accordance with an embodiment. In this example, the sensor device 102 is used in a hospital/clinic/medical care environment, where the sensor device 102 may be placed under the mattress or bed of the user, who may be a patient suffering from one or more health issues. The sensor device 102 can be used to monitor the body functions, cardiovascular functions, cardiac functions and ejection fraction of the patient in a non-intrusive, real-time continuous manner, without requiring any human intervention from nurses, doctors or healthcare professionals.

In an embodiment, the data capturing device 104 may be positioned in the vicinity of the patient, as depicted in the figure. In an embodiment, the connection between the sensor device 102 and the data capturing device 104 may be a wired or a wireless connection, depending on the environment and user requirement. Further, the data capturing device 104 may use an in-built battery source, or a wired battery source, for its functioning.

FIG. 4B depicts/illustrates an Energy Spectrum 402 for a user with Ejection Fraction (EF)>50%, in accordance with an embodiment. The EF computation unit 214 may determine that the user with EF>50% does not require immediate medical intervention, and may continue to monitor the user.

FIG. 4C depicts/illustrates an Energy Spectrum 404 for a user with EF<50%, in accordance with an embodiment. The EF computation unit 214 may determine that the user with EF<50% requires immediate medical intervention, and may generate one or more alerts to caretakers, nurses and healthcare professionals in order to provide timely medical intervention for the user.

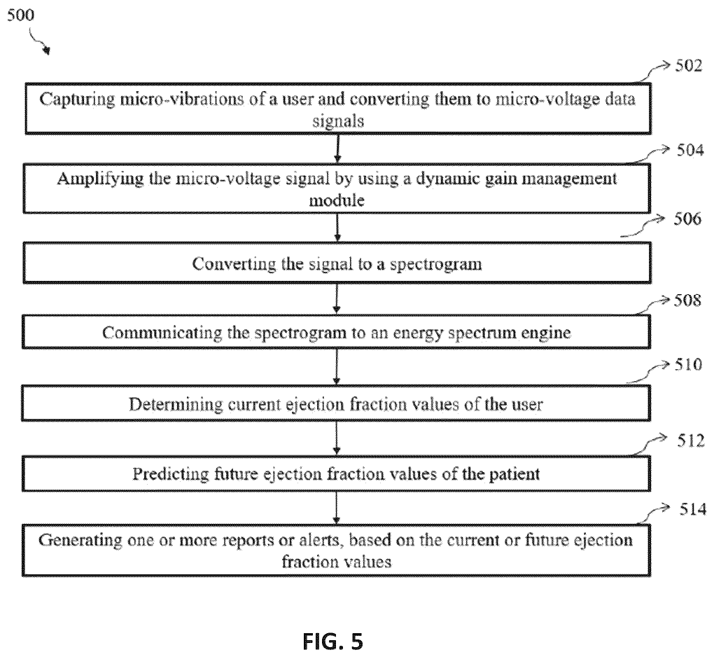

FIG. 5 illustrates a method 500 for non-intrusive monitoring and prediction of body function, in accordance with an embodiment.

The method begins with capturing micro-vibrations of a user and converting them to micro-voltage data signals, as depicted at step 502.

Subsequently, the method 500 discloses amplifying the micro-voltage signal by using a dynamic gain management module, as depicted at step 504.

Thereafter, the method 500 discloses converting the signal to a spectrogram, as depicted at step 506. Further, the method 500 discloses communicating the spectrogram to an energy spectrum engine, as depicted at step 508. The method 500 then discloses determining current ejection fraction values of the user, as depicted at step 510.

Further, the method 500 discloses predicting future ejection fraction values of the patient, as depicted at step 512. Subsequently, the method 500 discloses generating one or more reports or alerts, based on the current or future ejection fraction values, as depicted at step 514.

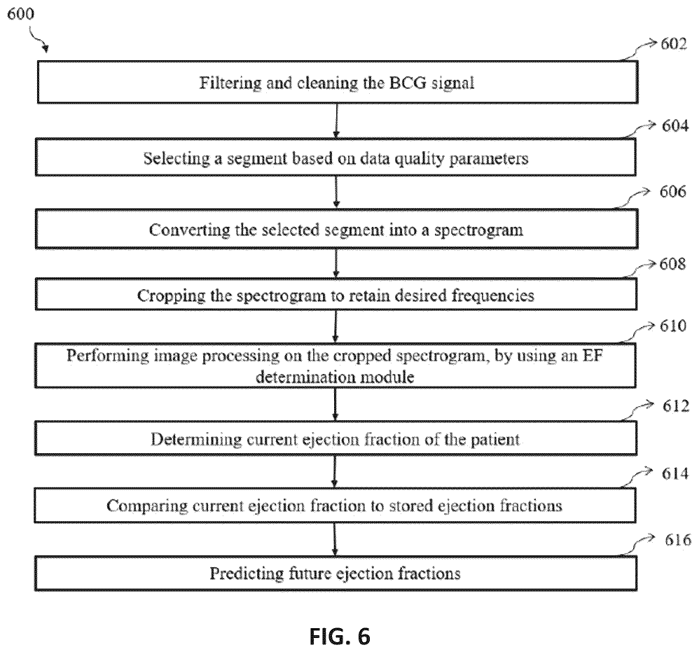

FIG. 6 illustrates a method 600 for non-intrusive prediction of ejection fraction, in accordance with an embodiment.

The method begins with filtering and cleaning the BCG signal, as depicted at step 602. The filtering and cleaning may involve analog and digital filtering methodology that are presently known or may be developed in the future. Subsequently, the method 600 discloses selecting a segment based on data quality parameters, as depicted at step 606. Thereafter, the method 600 discloses converting the selected segment into a spectrogram, as depicted at step 606.

Further, the method 600 discloses cropping the spectrogram to retain desired frequencies, as depicted at step 608. The method 600 discloses performing image processing on the cropped spectrogram, by using an EF determination module, as depicted at step 610.

Further, the method 600 discloses determining current ejection fraction of the patient, as depicted at step 612. The method 600 then discloses comparing current ejection fraction to stored ejection fractions, as depicted at step 608. Additionally, the method 600 discloses predicting future ejection fractions, as depicted at step 608.

In an embodiment, the disclosed system 100 and methods 500, 600 may be suitably implemented as a computer program to be implemented by a computing device. The method described herein may be typically implemented as a set of instructions to be executed by a computer, laptop, server, cloud server, or other similar computing devices.

In an embodiment, the set of program instructions may comprise a series of computer readable codes saved in a tangible storage comprising at least one of a computer readable storage medium such as a ROM, EPROM, flash drive, or hard disk; or may be transmitted to the computing device through wired or wireless means.

In an embodiment, the implementation of the disclosed system 100 and methods 500, 600 as a computer program may comprise an intangible form by using wireless communication.

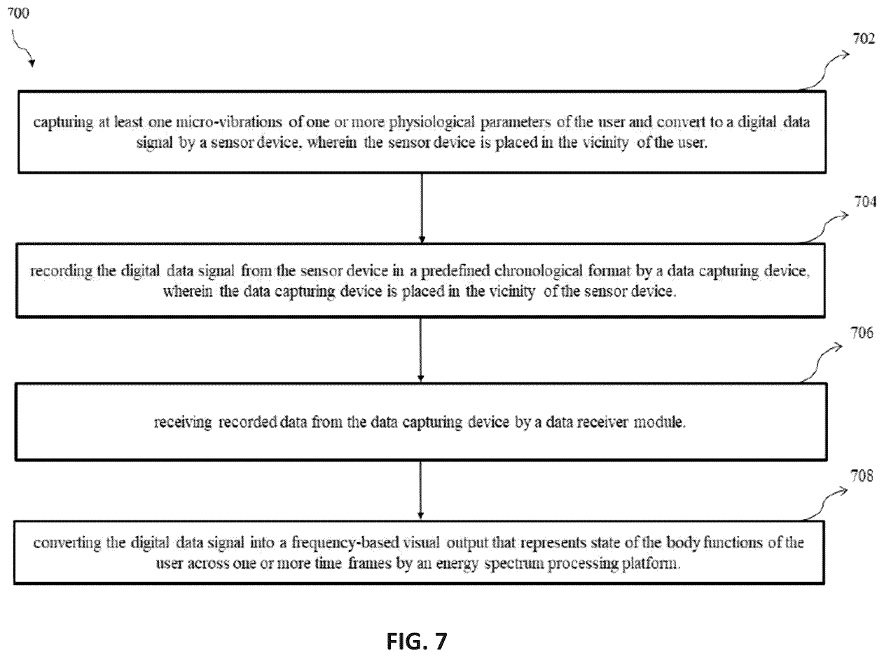

FIG. 7 illustrates a method 700 for a method for non-intrusive monitoring and prediction of body functions of a user. The method begins with capturing at least one micro-vibrations of one or more physiological parameters of the user and convert to a digital data signal by a sensor device 102, wherein the sensor device is placed in the vicinity of the user, as depicted at step 702. Subsequently, the method 700 discloses recording the digital data signal from the sensor device 102 in a predefined chronological format by a data capturing device 104, wherein the data capturing device 104 is placed in the vicinity of the sensor device 102, as depicted at step 704. Thereafter, the method 700 discloses receiving recorded data from the data capturing device 104 by a data receiver module, as depicted at step 706. Thereafter, the method 700 discloses converting the digital data signal into a frequency-based visual output that represents state of the body functions of the user across one or more time frames by an energy spectrum processing platform 108, as depicted at step 708.

The advantages of the current invention include providing a simple, autonomous and cost-effective system and method for non-intrusive monitoring and prediction of body functions, cardiovascular functions, cardiac functions and ejection fraction of a user.

An additional advantage is that the technology reduces the skill level required to test body, cardiovascular or cardiac outputs, as it does not require trained medical practitioners to analyze the BCG signal of the user to determine cardiac function and ejection fraction of the user.

Another additional advantage is that the disclosed system is less or non-intrusive and very comfortable when implemented in the vicinity of the user or patient. Additionally, the system can also be used in a daily or continuous manner in order to autonomously detect rapid changes in cardiac output and ejection fraction, without human intervention.

Applications of the current invention include remote patient monitoring and prediction of body functions, cardiovascular functions, cardiac functions and ejection fraction. The described system can be used in hospitals, clinics, camp set-ups, homes, humanitarian shelters, etc.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the scope of the embodiments as described here.

I/We claim:

1. A system for non-intrusive monitoring and prediction of body functions of a user, comprising:
a sensor device (102) placed in the vicinity of the user such that the sensor device is able to capture at least one micro-vibrations of one or more physiological parameters of the user and convert to a digital data signal;
a data capturing device (104) placed in the vicinity of the sensor device (102), the data capturing device configured to record the digital data signal from the sensor device (102) in a predefined chronological format;
a data receiver module (106) configured to receive recorded data from the data capturing device (104); and
an energy spectrum processing platform (108) configured to convert the digital data signal into a frequency-based visual output that represents state of the body functions of the user across one or more time frames.

2. The system as claimed in claim 1, wherein the at least one micro vibrations comprises at least one or more mechanical or force-base signals such as ballistocardiograph (BCG), seismo-cardiographs, and impedance signals associated with physiological parameters of the user's body.

3. The system as claimed in claim 1, wherein the data capturing device (104) consists of a conditioning unit (206) that is configured to determine the optimized signals for efficient detection of user body function, amplify and maximize the resolution of the digital signal.

4. The system as claimed in claim 3, wherein the optimized signal is communicated to the data receiver module (106) at regular intervals.

5. The system as claimed in claim 1, wherein the energy spectrum processing platform (108) is configured to compare the cardio pulmonary functions of the user with previously stored data regarding cardio pulmonary functions in the databases in order to predict future cardio pulmonary functions of the user.

6. The system as claimed in claim 1, wherein the energy spectrum processing platform (108) is configured to generate one or more reports and alerts based on the determined current and future body functions, cardiovascular functions, cardiac functions and cardio pulmonary functions based on the spectrogram.

7. The system as claimed in claim 1, wherein the energy spectrum processing platform (108) comprises an energy spectrum engine (212) configured to collect data from the data capturing device (104) and the sensor device (102) to analyze received data.

8. The system as claimed in claim 7, wherein the energy spectrum engine (212) comprises:
an EF computation unit (214) configured to compute the ejection fraction (EF) of a user based on the energy spectrum of the body function data;
an energy spectrum features unit (216) configured to process the spectrogram by adding one or more layers of filtering and analysis; and
a database (218) configured to store one or more micro-voltage digital signals in a predetermined data storage format.

9. The system as claimed in claim 1, wherein the digital data signals may comprise micro-voltages in a range of 0.1V to 3.5V.

10. The system as claimed in claim 1, comprises a user device (110) configured to continuously view and monitor user body functions, cardiovascular functions, and cardiac functions such as cardio-pulmonary, and receive one or more reports, and alerts.

11. The system as claimed in claim 1, comprises a communication network (112) to bridge the gap between the data capturing device (104) and the data receiver module (106).

12. A method for non-intrusive monitoring and prediction of body functions of a user, comprising:
capturing at least one micro-vibrations of one or more physiological parameters of the user and converting to a digital data signal by a sensor device (102), wherein the sensor device is placed in the vicinity of the user;
recording the digital data signal from the sensor device (102) in a predefined chronological format by a data capturing device (104), wherein the data capturing device (104) is placed in the vicinity of the sensor device (102);
receiving recorded data from the data capturing device (104) by a data receiver module (106); and
converting the digital data signal into a frequency-based visual output that represents state of the body functions of the user across one or more time frames by an energy spectrum processing platform (108).

13. The method as claimed in claim 12, determining a conditioning unit (206) in the data capturing device (104) to the optimized signals for efficient detection of user body function, amplify and maximize the resolution of the digital signal

14. The method as claimed in claim 13, communicating the optimized signal to the data receiver module (106) at regular intervals.

15. The method as claimed in claim 12, comparing the cardio pulmonary functions of the user with previously stored data regarding cardio pulmonary functions in the databases, in order to predict future cardio-pulmonary functions of the user, by the energy spectrum processing platform (108).

16. The method as claimed in claim 12, comprising configuring the energy spectrum processing platform (108) to collect data from the data capturing device (104) and the sensor device (102) to analyze received data by an energy spectrum engine (212).

17. The method as claimed in claim 16, comprising the energy spectrum engine (212) comprises:
configuring to compute the ejection fraction (EF) of a user based on the energy spectrum of the body function data by an EF computation unit (214);
processing the spectrogram by adding one or more layers of filtering and analysis by an energy spectrum features unit (216);
storing one or more micro-voltage digital signals in a predetermined data storage format by a database (218).

18. The method as claimed in claim 12, viewing continuously and monitoring the user body functions, cardiovascular functions, and cardiac functions such as ejection fraction, and receiving one or more reports, and alerts by a user device (110).

19. The method as claimed in claim 12, comprising bridging the gap between the data capturing device (104) and the data receiver module (106) by a communication network (112).
